(19) United States
(12) Patent Application Publication
(10) Pub. No.: US 2007/0009443 A1
(43) Pub. Date: Jan. 11, 2007

(54) Method for inhibiting occupation by rhinovirus of cell in the nasal membrane

(76) Inventor: Charles B. Hensley, Redondo Beach, CA (US)

Correspondence Address: TOD R NISSLE, PO BOX 55630, PHOENIX, AZ, 85078, US

(21) Appl. No.: 11/175,913

(22) Filed: Jul. 5, 2005

(51) Int. Cl. A61L 9/04; A61K 36/18

(52) U.S. Cl. 424/45; 424/725

(57) ABSTRACT

A composition and method for modifying the composition of a cell in a nasal epithelial membrane to improve the ability of the cell to defend against rhinovirus.

This invention relates to methods and compositions for penetrating a cell to modify the functioning of the cell.

More particularly, the invention relates to a method and composition for modifying the functioning of a cell in the nasal membrane to inhibit the production of NFKappaB, to inhibit the acidification of the cell, and to inhibit the uncoating of the cell.

The common cold is one of the most frequently occurring human illnesses and is responsible for substantial morbidity and economic loss. A variety of compositions have been developed to treat the common cold.

I have discovered a novel composition and method for interfering with the use by rhinovirus of a cell in the nasal membrane. The composition contacts the nasal membrane, enters a cell in the nasal membrane, inhibits the production of NFKappaB, and inhibits the acidification and uncoating of the cell. The composition includes from 40% to 99.999% by weight water and from 0.001% to 30% by weight of a reducing agent that inhibits the production of NFKappaB in a nasal cell. The composition can also include 0.001% to 30% by weight of catechins and/or epicatechins to inhibit the acidifcation of the cell; include 0.001% to 30% by weight of a composition the enhances the ability of the reducing agent and catechins and epicatechins to penetrate and enter a nasal cell; include from 0.001% to 2.0% by weight of copper and/or magnesium, preferably in an ionic state; and, include a foaming agent that produces a gas when the composition is administered in the nasal cavity of an individual. The composition is preferably, but not necessarily, administered by spraying the composition into the nasal cavity for direct contact with the nasal epithelial membrane. Minor effective amounts of preservatives, coloring agents, scents, etc. can be incorporated as desired in the composition.

Glutathione is one preferred reducing agent that is believed to inhibit the production of NFKappaB. Other natural reducing agents include ascorbic acid, N-Acetylcysteine, superoxide dismutase, Resveratrol. Caffeic acid, Coenzyme Q10 and Alpha-tocopherol. When the production of NFKappaB is inhibited, the synthesis of ICAM in a nasal cell also is inhibited.

Liposomes are the preferred permeation enhancers. Other natural permeation enhancers include ascorbic acid, terpenes and flavonoids. Liposomes enhance the ability of reducing agents, catechins, and epicatechins to enter a nasal cell.

The presently preferred catechins are found in green tea or green tea extract (GTE) and include (−)-epigallocatechin gallate (EGCG), (−) epigalloatechin (EGC), (−) epicatechin gallate (ECG), (+)-epicatechin (EC), (−)-gallocatechin gallate (GCG), and catechin. When these catechins are incorporated in the composition of the invention, and the composition penetrates a nasal cell, it is believed that acidification of the cell is inhibited, which also inhibits the uncoating of the cell by a virus that is attempting to enter the cell. Although not a requisite, compositions prepared in accordance with the invention include GTE and each of catechins found therein because it is theorized that the different forms of catechins enable the composition to better prevent acidification of a nasal cell. In addition to catechins and epicatetchins, other natural anti-acidification enhancers include ephedrae herba.

The source of copper and/or magnesium preferably is copper sulfate, magnesium sulfate, or magnesium chloride. It is surmised that some ionic copper or magnesium released in the nasal cavity functions as ICAM-1 blockers by binding to viral ICAM-1 receptors and inhibiting rhinovirus from binding to and infecting nasal mucosal cells.

The composition of the invention can be administered any desired number of times, but preferably is administered at least once a day for several days or until the symptoms associated with a common cold have abated. The size of the dose of the composition can also vary as desired, but typically is in the range of about ¼ to 1/25 of an ounce, preferably about ⅕ to 1/10 of an ounce.

The following examples depict the presently preferred embodiments of the invention for the purposes of illustrating the practice thereof and not by way of limitation of the scope of the invention. In the examples, all proportions are by weight percent, unless otherwise noted.

### EXAMPLE 1

One hundred ounces of an aqueous composition containing 98.5% by weight purified water, 0.5% by weight glutathione as a reducing agent, and 1.0% by weight liposomes as a permeation enhancer is prepared by admixing the water, glutathione, and liposomes at room temperature.

### EXAMPLE 2

One hundred ounces of an aqueous composition containing 98.0% by weight purified water, 0.5% by weight glutathione, 0.5% GTE as an anti-acidification agent, and 1.0% by weight liposomes is prepared by admixing the water, glutathione, and liposomes at room temperature.

### EXAMPLE 3

One hundred ounces of a liquid composition containing 98.0% by weight purified water, 0.5% by weight glutathione, 0.5% by weight copper sulfate as an ICAM-1 blocker, and 1.0% by weight liposomes is prepared by admixing the water, glutathione, and liposomes at room temperature. If desired, ethanol can be utilized in place of or in combination with water to constitute the 98% by weight portion of the afore-mentioned composition and other liquid compositions prepared in accordance with the invention. The proportion of water to alcohol can vary as desired.

### EXAMPLE 4

Example 1 is repeated except ascorbic acid is utilized as a reducing agent instead of glutathion.

### EXAMPLE 5

Example 1 is repeated, except N-acetylcysteine is used as a reducing agent in place of glutathion.

### EXAMPLE 6

Example 2 is repeated, except ascorbic acid is used as a reducing agent in place of glutathion.

### EXAMPLE 7

Example 3 is repeated, except N-acetylcysteine is used as a reducing agent in place of glutathion.

### EXAMPLE 8

Example 2 is repeated, except ephedrae herba is used in place of GTE.

### EXAMPLE 9

Example 1 is repeated, except a flavonoid is used in place of liposomes.

### EXAMPLE 10

Example 2 is repeated, except ephedrae herba is utilized as an anti-acidification agent in place of GTE.

### EXAMPLE 11

Example 3 is repeated, except magnesium sulfate is utilized as an ICAM-1 blocker in place of copper sulfate.

### EXAMPLE 12

Examples 1 to 11 are repeated, except that the weight percent of the reducing agent utilized is 0.05% instead of 0.5%, and that the weight percent of water is increased by 0.45%.

### EXAMPLE 13

Examples 1 to 11 are repeated, except that the weight percent of the reducing agent utilized is 5% instead of 0.5%, and that the weight percent of water is decreased by 4.5%.

### EXAMPLE 14

Examples 2, 6, 8, 10 are repeated, except that the weight percent of the anti-acidification agent utilized is 0.05% instead of 0.5%, and that the weight percent of water is increased by 0.45%.

### EXAMPLE 15

Examples 2, 6, 8, 10 are repeated, except that the weight percent of the anti-acidification agent utilized is 5% instead of 0.5%, and that the weight percent of water is decreased by 4.5%.

### EXAMPLE 16

Examples 3, 7, 11 are repeated, except that the concentration of ICAM-1 blocker is 0.05% instead of 0.5% and the weight percent of water is increased by 0.45%.

### EXAMPLE 17

Examples 3, 7, 11 are repeated, except that the concentration of the ICAM-1 blocker is 2.0% instead of 0.5% and the weight percent of water is decreased by 1.5%.

### EXAMPLE 18

200 microliters of the aqueous solution of Example 1 is sprayed in the nasal cavity of a first eighteen year old female Caucasian who has been experiencing mild cold symptoms for a day. After three hours the individual notices a reduction in the severity of her cold symptoms. A second eighteen year old Caucasian girl also has been experiencing mild cold symptoms for one day. The composition of the invention is not administered to the second girl, nor is any other medication. After three hours, the second girl does not notice any reduction in the severity of her cold symptoms.

### EXAMPLE 19

200 microliters of the aqueous solution of Example 1 is sprayed in the nasal cavity of a first fifteen year old female Japanese who has been experiencing mild cold symptoms for a day. After three hours the individual notices a reduction in the severity of her cold symptoms. A second fifteen year old Japanese girl also has been experiencing mild cold symptoms for one day. The composition of the invention is not administered to the second girl, nor is any other medication. After three hours, the second girl does not notice any reduction in the severity of her cold symptoms.

### EXAMPLE 20

200 microliters of the aqueous solution of Example 1 is sprayed in the nasal cavity of a first fifty-six year old African-American male who has been experiencing mild cold symptoms for a day. After three hours the individual notices a reduction in the severity of her cold symptoms. A second fifty-six year old African-American male also has been experiencing mild cold symptoms for one day. The composition of the invention is not administered to the second African-American male, nor is any other medication. After three hours, the second African-American male does not notice any reduction in the severity of his cold symptoms.

### EXAMPLE 21

Examples 18 to 21 are repeated using the composition of Example 2 instead of the composition of Example 1. Similar results are obtained.

### EXAMPLE 22

Examples 18 to 21 are repeated using the composition of Example 2 instead of the composition of Example 1. Similar results are obtained.

### EXAMPLE 23

Examples 18 to 21 are repeated using the composition of Example 3 instead of the composition of Example 1. Similar results are obtained.

### EXAMPLE 24

Examples 18 to 21 are repeated using the composition of Example 4 instead of the composition of Example 1. Similar results are obtained.

### EXAMPLE 25

Examples 18 to 21 are repeated using the composition of Example 5 instead of the composition of Example 1. Similar results are obtained.

### EXAMPLE 26

Examples 18 to 21 are repeated using the composition of Example 6 instead of the composition of Example 1. Similar results are obtained.

### EXAMPLE 27

Examples 18 to 21 are repeated using the composition of Example 7 instead of the composition of Example 1. Similar results are obtained.

### EXAMPLE 28

Examples 18 to 21 are repeated using the composition of Example 8 instead of the composition of Example 1. Similar results are obtained.

### EXAMPLE 29

Examples 18 to 21 are repeated using the composition of Example 9 instead of the composition of Example 1. Similar results are obtained.

### EXAMPLE 30

Examples 18 to 21 are repeated using the composition of Example 10 instead of the composition of Example 1. Similar results are obtained.

### EXAMPLE 31

Examples 18 to 21 are repeated using the composition of Example 11 instead of the composition of Example 1. Similar results are obtained.

### EXAMPLE 32

Examples 18 to 21 are repeated using the composition of Example 12 instead of the composition of Example 1. Similar results are obtained.

### EXAMPLE 33

Examples 18 to 21 are repeated using the composition of Example 13 instead of the composition of Example 1. Similar results are obtained.

### EXAMPLE 34

Examples 18 to 21 are repeated using the composition of Example 14 instead of the composition of Example 1. Similar results are obtained.

### EXAMPLE 35

Examples 18 to 21 are repeated using the composition of Example 15 instead of the composition of Example 1. Similar results are obtained.

### EXAMPLE 36

Examples 18 to 21 are repeated using the composition of Example 16 instead of the composition of Example 1. Similar results are obtained.

### EXAMPLE 37

Examples 18 to 21 are repeated using the composition of Example 17 instead of the composition of Example 1. Similar results are obtained.

### EXAMPLE 38

Examples 18 to 37 are repeated except fifty microliters of aqueous solution are utilized instead 200 microliters. Similar results are obtained.

### EXAMPLE 39

Examples 18 to 37 are repeated except 300 microliters of aqueous solution are utilized instead 200 microliters. Similar results are obtained.

### EXAMPLE 40

Examples 18 to 37 are repeated except 100 microliters of aqueous solution are utilized instead 200 microliters. Similar results are obtained.

### EXAMPLE 41

Examples 1 to 17 are repeated except the weight percent of water is reduced by 50% and ethanol is substituted for the water eliminated from the composition. Consequently, if the liquid composition is 98% by weight water, the liquid composition becomes 49% by weight water and 49% by weight ethanol. Similar results are obtained.

### EXAMPLE 42

Examples 18 to 37 are repeated utilizing the water-alcohol compositions of Example 41. Similar results are obtained.

### EXAMPLE 43

Examples 1 to 17 are repeated except the weight percent of water is reduced by 80% and ethanol is substituted for the water eliminated from the composition. Consequently, if the liquid composition is 98% by weight water, the liquid composition becomes about 20% by weight water and 68% by weight ethanol. Similar results are obtained.

### EXAMPLE 44

Examples 18 to 37 are repeated utilizing the water-alcohol compositions of Example 43. Similar results are obtained.

The liquid compositions of the invention can be modified in any desired many to foam when inserted in a nasal cavity. By way of example, ant not limitation, the liquid composition can be carbonated, placed in a pressurized container and cooled such that carbon dioxide gas is released when the composition is applied to a nasal cavity.

Having described my invention in such terms as to enable those skilled in the art to use the invention, and having described the presently preferred embodiments thereof, I claim:

1. A method of delivering to a cell in a nasal epithelial membrane in a nasal cavity a reducing agent to relieve symptoms of a common cold, comprising the steps of
(a) providing a liquid delivery composition comprising
(i) at least one natural reducing agent to inhibit the expression if ICAM in a nasal cell,
(ii) at least one permeation enhancer to facilitate the introduction of said reducing agent in the nasal cell, and
(iii) a liquid carrier;
(b) applying said liquid composition in contact with the nasal epithelial membrane in the nasal cavity.

2. A method of delivering to a cell in a nasal epithelial membrane in a nasal cavity a reducing agent to relieve symptoms of a common cold, comprising the steps of
(a) providing a liquid delivery composition comprising
(i) at least one natural reducing agent to inhibit the expression if ICAM in a nasal cell,
(ii) at least one anti-acidification agent to inhibit the acidification of a nasal cell,
(iii) at least one permeation enhancer to facilitate the introduction of said reducing agent and said anti-acidification agent in the nasal cell, and
(iv) a liquid carrier; and,
(b) applying said liquid composition in contact with the nasal epithelial membrane in the nasal cavity.
